(19) United States
(12) Patent Application Publication
(10) Pub. No.: US 2021/0315991 A1
(43) Pub. Date: Oct. 14, 2021

(54) COMBINATION INCLUDING A CPG-C TYPE OLIGONUCLEOTIDE AND A PD-1 ANTAGONIST FOR TREATING BREAST CANCER

(71) Applicants: Surefire Medical, Inc. d/b/a TriSalus Life Sciences, Westminster, CO (US); The Regents of the University of California, Oakland, CA (US); H. Lee Moffitt Cancer Center and Research Institute, Inc., Tampa, FL (US)

(72) Inventors: Robert S. Janssen, Westminster, CO (US); Erick Gamelin, Westminster, CO (US); Laura J. Esserman, Westminster, CO (US); Amy Jo Chien, Westminster, CO (US); Hetem Soliman, Westminster, CO (US)

(73) Assignees: Surefire Medical, Inc. d/b/a TriSalus Life Sciences, Westminster, CO (US); The Regents of the University of California, Oakland, CA (US); H. Lee Moffitt Cancer Center and Research Institute, Inc., Tampa, FL (US)

(21) Appl. No.: 17/229,856

(22) Filed: Apr. 13, 2021

Related U.S. Application Data
(63) Continuation of application No. PCT/US2019/055941, filed on Oct. 11, 2019.
(60) Provisional application No. 62/745,421, filed on Oct. 14, 2018.

(51) Int. Cl. A61K 39/39; A61P 35/00; A61K 39/395; A61K 31/337; A61K 9/00; A61K 31/704; A61K 31/675

(52) U.S. Cl. CPC A61K 39/39; A61P 35/00; A61K 39/3955; A61K 2039/55561; A61K 9/0019; A61K 31/704; A61K 31/675; A61K 31/337

(57) ABSTRACT

The present disclosure describes combination therapies for breast cancer comprising an oligonucleotide toll-like receptor 9 agonist and a PD-1 antagonist. In particular, the present disclosure describes combinations of a CpG-C type oligonucleotide and an anti-PD-1 antibody for the treatment of breast cancer. The combination may further comprise a taxane chemotherapeutic agent, in the presence or absence of a corticosteroid.

### CROSS-REFERENCE TO RELATED APPLICATIONS

This application claims is a Continuation of International Application No. PCT/US2019/055941, filed Oct. 11, 2019, published on Apr. 23, 2020 under Publication No. WO 2020/081398, which benefit of U.S. Provisional Application No. 62/745,421, filed Oct. 14, 2018, the disclosure of which are hereby incorporated by reference in its entireties.

### SUBMISSION OF SEQUENCE LISTING ON ASCII TEXT FILE

The instant application contains a Sequence Listing which has been submitted electronically in ASCII format and is hereby incorporated by reference in its entirety. Said ASCII copy, created on Jun. 21, 2021, is named A372-625_1_SL.txt and is 13,430 bytes in size.

### FIELD

The present disclosure describes combination therapies for breast cancer comprising an oligonucleotide toll-like receptor 9 agonist and a PD-1 antagonist. In particular, the present disclosure describes combinations of a CpG-C type oligonucleotide and an anti-PD-1 antibody for the treatment of breast cancer. The combination may further comprise a taxane chemotherapeutic agent, in the presence or absence of a corticosteroid.

### BACKGROUND

The programmed cell death protein 1 (PD-1) is predominantly expressed on activated T and B lymphocytes. Binding of PD-1 to the programmed death-ligand 1 (PD-L1) acts as an immune checkpoint resulting in down regulation of T cell mediated immune responses. In this way, PD-L1 expression by tumors and infiltrating myeloid cells is a mechanism through which tumors are able to evade an anti-tumor T cell response. Consequently, therapeutic agents that block the PD-1/PD-L1 pathway have been developed for use in cancer immunotherapy (Gong et al., J Immuno Therapy Cancer, 6:8, 2018). Since 2014, several PD-1 antagonists have been approved by the U.S. Food and Drug Administration for the treatment of multiple tumor types.

Pembrolizumab and nivolumab are anti-human PD-1 antibodies approved for treatment of metastatic melanoma. Although pembrolizumab and nivolumab have shown a high rate of durable response in some metastatic melanoma patients, other patients fail to respond or relapse after an initial response. Recent studies have found that response to anti-PD-1 therapy is contingent on the presence of tumor infiltrating lymphocytes (Tumeh et al., Nature, 515:568-571, 2014). In addition, the degree of T-cell infiltration in melanoma is associated with the presence of type I interferon activation (Bald et al., Cancer Discovery, 4:674-687, 2014), suggesting that an immune rich tumor environment is necessary for a response to PD-1 antagonists.

Toll-like receptors (TLRs) play an important role in the immune system. In humans, TLR9 is expressed in endosomes of dendritic cells and B cells (Melisi et al., Biomedicines, 2:211-228, 2014). TLR9 is activated by unmethylated CpG motifs in DNA leading to initiation of an innate immune response and stimulation of an adaptive immune response. Synthetic oligodeoxynucleotides containing unmethylated CpG motifs (CpG-ODNs) have been found to induce maturation of dendritic cells and to promote differentiation of B cells. Several CpG-ODNs have been developed as TLR9 agonists for cancer therapy (Melisi et al., Biomedicines, 2:211-228, 2014; and Shirota et al., Vaccines, 3:390-407, 2015). Although results of preclinical and phase II trials were promising, systemic administration of a CpG-ODN did not improve survival of patients with non-small cell lung cancer when combined with a chemotherapy regimen (Schmidt, Nature Biotechnology, 25:825-826, 2007). The route of administration of polynucleotide TLR9 agonists has since been shown to be critical, with intratumoral injection resulting in superior antitumor immune responses than intravenous injection (Lou et al., J Immunother, 34:279-288, 2011).

According to the American Cancer Society in the United States alone over 250,000 new cases of breast cancer will be diagnosed in women, and over 40,000 women will die of breast cancer in 2018. Preoperative chemotherapy (also known as neoadjuvant therapy) is widely employed in treatment regimens for locally advanced breast cancer as it promotes disease downsizing and conservation of breast tissue (Bartsh et al., Memo, 11:199-203, 2018). In order to improve the efficacy of neoadjuvant chemotherapy, a variety of therapeutic agents are being tested in combination with standard chemotherapy regimens. Modest overall response rates were observed in initial clinical trials employing pembrolizumab monotherapy in previously treated breast cancer patients (Nanda et al., J Clin Oncol, 34:2460-2467, 2016; and Rugo et al., Clin Caner Res, 24:2804-2811, 2018). Even so, the addition of pembrolizumab to a standard chemotherapy regimen was recently found to improve pathologic complete response (pCR) rates in some groups of breast cancer patients (Nanda et al., J Clin Oncol, 35(15_suppl): 506, 2017).

Dexamethasone is typically administered as a premedication to prevent infusion related hypersensitivity to chemotherapy. Dexamethasone and other corticosteroid medications have anti-inflammatory and immunosuppressant effects. As such, corticosteroid premedication may counteract the anti-tumor response induced by intratumoral injection of a CPG-ODN alone or in combination with a PD-1 antagonist. Thus, there remains a need in the art for identification of improved treatment regimens for pre-operative, post-operative, and inoperative breast cancer, particularly for regimens involving chemotherapy and corticosteroid premedication.

### BRIEF SUMMARY

The present disclosure provides methods for treating breast cancer comprising administering an oligonucleotide toll-like receptor 9 (TLR) agonist in combination with a PD-1 antagonist to a mammalian subject in need thereof. In particular, the present disclosure provides a method of treating breast cancer in a mammalian subject in need thereof, comprising intratumoral administration of a CpG-C type oligonucleotide and intravenous administration of an anti-PD-1 antibody. In some embodiments, the methods further comprise intravenous administration of a taxane chemotherapeutic agent, optionally following administration of a corticosteroid. In some embodiments, the mammalian subject is a human patient with HER2-negative breast cancer.

Moreover, the present disclosure provides a composition comprising a CpG-C type oligonucleotide for use in a method of treating breast cancer in a mammalian subject in need thereof, the method comprising intratumoral administration of a CpG-C type oligonucleotide and intravenous administration of an anti-PD-1 antibody. In some embodiments, the methods further comprise intravenous administration of a taxane chemotherapeutic agent, optionally following administration of a corticosteroid. In some embodiments, the mammalian subject is a human patient with HER2-negative breast cancer.

Additionally, the present disclosure provides a medicament comprising a CpG-C type oligonucleotide for use in a method of treating breast cancer in a mammalian subject in need thereof, the method comprising intratumoral administration of a CpG-C type oligonucleotide and intravenous administration of an anti-PD-1 antibody. In some embodiments, the methods further comprise intravenous administration of a taxane chemotherapeutic agent, optionally following administration of a corticosteroid. In some embodiments, the mammalian subject is a human patient with HER2-negative breast cancer.

### DETAILED DESCRIPTION

The present disclosure relates to combination therapies for breast cancer. In particular, the present disclosure provides a CpG-C type oligonucleotide (CpG-C ODN) in combination with a PD-1 antagonist for treatment of breast cancer. In some embodiments, the methods further comprise intravenous administration of a taxane chemotherapeutic agent, optionally following administration of a corticosteroid. In some embodiments, the mammalian subject is a human patient with HER2-negative breast cancer.

### I. General Methods and Definitions

As used herein and in the appended claims, the singular forms “a,” “an,” and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 200 mg pembrolizumab refers to 180 mg to 220 mg pembrolizumab and includes 200 mg pembrolizumab).

As used interchangeably herein, the terms “polynucleotide,” “oligonucleotide” and “nucleic acid” include single-stranded DNA (ssDNA), double-stranded DNA (dsDNA), single-stranded RNA (ssRNA) and double-stranded RNA (dsRNA), modified oligonucleotides and oligonucleosides, or combinations thereof. Polynucleotides are polymers of nucleosides joined, generally, through phosphodiester linkages, although alternate linkages, such as phosphorothioate esters may also be used. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. The four nucleoside units (or bases) in RNA are called adenosine, guanosine, uridine and cytidine. A nucleotide is a phosphate ester of a nucleoside.

The term “palindromic sequence” or “palindrome” refers to a nucleic acid sequence that is an inverted repeat, e.g., ABCDD′C′B′A′, where the bases, e.g., A, and A′, B and B′, C and C′, D and D′, are capable of forming Watson-Crick base pairs. Such sequences may be single-stranded or may form double-stranded structures or may form hairpin loop structures under some conditions. For example, as used herein, “an 8 base palindrome” refers to a nucleic acid sequence in which the palindromic sequence is 8 bases in length, such as ABCDD′C′B′A′. A palindromic sequence may be part of a polynucleotide that also contains non-palindromic sequences. A polynucleotide may contain one or more palindromic sequence portions and one or more non-palindromic sequence portions. Alternatively, a polynucleotide sequence may be entirely palindromic. In a polynucleotide with more than one palindromic sequence portions, the palindromic sequence portions may or may not overlap with each other.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The terms “CpG” and “CG” are used interchangeably herein to refer to, unless stated otherwise, a cytosine and guanine separated by a phosphate. These terms refer to a linear sequence as opposed to base-pairing of cytosine and guanine. The polynucleotides of the present disclosure contain at least one unmethylated CpG dinucleotide. That is the cytosine in the CpG dinucleotide is not methylated (i.e., is not 5-methylcytosine).

“CpG polynucleotides” or “CpG oligonucleotides” of the present disclosure are nucleic acids from 12 to 40 nucleotides in length, which comprise one or more unmethylated CG dinucleotides. In some preferred embodiments, the oligonucleotide is an oligodeoxynucleotide (ODN). In some preferred embodiments, the CpG ODN includes a TCN or TCG at its 5′ end, which imparts the ability to stimulate B cells. In some embodiments, the CpG ODN includes a CG-containing palindrome, which imparts the ability to induce human plasmacytoid dendritic cell (PDC) maturation and secretion of high levels of type I interferons (e.g., IFN-α, IFN-γ, etc.). In some embodiments, the ODN is at least (lower limit) 12, 13, 14, 15, 16, 17, 18, 19, 20, 22, 24, 26, 28, 30, 32, 34, 36, or 38 nucleotides in length. In some embodiments, the ODN is at most (upper limit) 40, 38, 36, 34, 32, 30, 28, 26, 24, 22 or 20 nucleotides in length.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response.

“Inhibition” of a response or parameter includes blocking and/or suppressing that response or parameter when compared to otherwise same conditions except for a parameter of interest, or alternatively, as compared to another condition (e.g., decrease in PD-1-signaling in the presence of a PD-1 ligand and a PD-1 antagonist as compared to the presence of the PD-1 ligand in the absence of the PD-1 antagonist). For example, “inhibition” of an immune response means a decrease in the response.

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., CpG-C type ODN) effective to “treat” a disease or disorder in a subject (e.g., a mammal such as a human). In the case of a combination therapy, an “effective amount” refers to the amounts of the active agents of the combination (e.g., CpG-C type ODN and PD-1 antagonist), which together are effective to “treat” a disease or disorder in a subject. An “effective amount” or an “amount sufficient” of an agent or agents may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

### II. CpG-C Type Oligonucleotides

TLR9 agonists suitable for the treatment methods, medicaments and uses of the present disclosure include oligodeoxynucleotides containing an unmethylated CpG dinucleotide. In some preferred embodiments, the CpG-containing oligodeoxynucleotide (CpG-ODN) is a CpG-C type ODN (CpG-C ODN). CpG-C ODNs have the ability to stimulate B cells, induce plasmacytoid dendritic cell (PDC) maturation and cause secretion of high levels of type I interferons (e.g., IFN-α, IFN-γ, etc.). In some preferred embodiments, the CpG-C ODN comprises:

5′-TCNq(X1X2CGX2′X1′CG)rNs-3′ (SEQ ID NO:1), wherein each N is an independently selected nucleoside; X1 and X1′ are self-complementary and X2 and X2′ are self-complementary; q=1 or 2; r=1, 2, 3 or 4; and s=1 to 29. The sequences of representative CpG-C ODN molecules are described in U.S. Pat. Nos. 7,745,606, 8,158,768, 8,871,732, and 9,422,564. The CpG-C ODN molecules of the present disclosure include at least one, two or three internucleotide phosphorothioate ester linkages. In some embodiments, when a plurality of CpG-C ODN molecules are present in a pharmaceutical composition comprising at least one excipient, both stereoisomers of the phosphorothioate ester linkage are present in the plurality of CpG-C ODN molecules. In one embodiment, all of the internucleotide linkages of the CpG-C ODN are phosphorothioate linkages. A phosphorothioate backbone refers to all of the internucleotide linkages of the CpG-C ODN being phosphorothioate (PS) linkages.

The CpG-C ODNs described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In one embodiment, the CpG-C type ODNs are in the ammonium, sodium, lithium, or potassium salt form. In one preferred embodiment, the CpG-C ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconstituted in sterile water, saline or a pharmaceutically acceptable buffer before administration.

In some preferred embodiments, the CpG-C ODN consists of: 5′-TCNDyAACGTTCGAACGTTCGAANx-3′ (SEQ ID NO:2) or 5′-TCGDyAACGTTCGAACGTTCGAANx-3′ (SEQ ID NO:3), wherein each N is an independently selected nucleoside; D is G, A or T; y=0 or 1; and x=0 to 19. Exemplary CpG-C ODNs comprise a sequence selected from the group consisting of:

(SEQ ID NO: 4)
5′-TCG AACGTTCGAACGTTCGAA CGTTCGAAT-3′;
(SEQ ID NO: 5)
5′-TCG AACGTTCGAACGTTCGAA CGTT-3′;
(SEQ ID NO: 6)
5′-TCG AACGTTCGAACGTTCGAA TTTT-3′;
(SEQ ID NO: 7)
5′-TCGTAACGTTCGAACGTTCGAA CGTTA-3′;
(SEQ ID NO: 8)
5′-TCGTAACGTTCGAACGTTCGAA CGTT-3′;
(SEQ ID NO: 9)
5′-TCGTAACGTTCGAACGTTCGAA CGT-3′;
(SEQ ID NO: 10)
5′-TCGTAACGTTCGAACGTTCGAA CG-3′;
(SEQ ID NO: 11)
5′-TCGTAACGTTCGAACGTTCGAA C-3′;
and
(SEQ ID NO: 12)
5′-TCGTAACGTTCGAACGTTCGAA-3′.

In a preferred embodiment, the CpG-C ODN comprises:

(SEQ ID NO: 4)
5′-TCG AACGTTCGAACGTTCGAA CGTTCGAAT-3′.

The activity of a candidate TLR9 agonist may be assessed in vitro by measuring interferon-alpha secretion by human peripheral blood mononuclear cells (PBMC) with an enzyme linked immunosorbent assay (ELISA). In particular, human PBMC are contacted with a series of graded doses of the candidate TLR9 agonist (and controls) for a period of about 24 hours. The EC₅₀ value (in nM) and a max production level (in pg/mL) are then determined from a dose-response curve.

### III. PD-1 Antagonists

PD-1 antagonists suitable for the treatment methods, medicaments and uses of the present disclosure include any chemical compound or biological molecule that blocks binding of PD-L1 expressed on a cancer cell to PD-1 expressed on an immune cell (T cell, B cell or NKT cell). Alternative names or synonyms for PD-1 and its ligands include: PDCD1, PD1, CD279 and SLEB2 for PD-1; PDCD1L1, PDL1, B7H1, B7-4, CD274 and B7-H for PD-L1. In any of the treatment methods, medicaments and uses of the present disclosure in which a human subject is being treated, the PD-1 antagonist blocks binding of human PD-L1 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists suitable for any of the treatment methods, medicaments and uses of the present disclosure include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and are suitable for the treatment methods, medicaments and uses of the present disclosure, are described in US 2018/0169229. Specific anti-human PD-1 mAbs useful as the PD-1 antagonist in the treatment methods, medicaments and uses of the present disclosure include pembrolizumab (MK-3475), nivolumab (BMS-936558 or MDX-1106), and cemiplimab (REGN2810). In some embodiments, the anti-human PD-1 mAb is a biosimilar of pembrolizumab, nivolumab, or cemiplimab.

In some embodiments, the anti-human PD-1 monoclonal antibody is a pembrolizumab variant which comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs (e.g., the variant positions are located in the framework regions or the constant region). In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

Examples of mAbs that bind to human PD-L1, and are suitable for the treatment methods, medicaments and uses of the present disclosure are described in US 2018/0169229. Specific anti-human PD-L1 mAbs useful as the PD-1 antagonist in the treatment methods, medicaments and uses of the present disclosure include atezolizumab (MPDL3280A), durvalumab (MEDI4736), and avelumab (MSB0010718C). In some embodiments, the anti-human PD-L1 mAb is a biosimilar of atezolizumab, durvalumab, or avelumab.

Other PD-1 antagonists suitable for the treatment methods, medicaments and uses of the present disclosure include an immunoadhesin that specifically binds to PD-1 or PD-L1, and preferably specifically binds to human PD-1 or human PD-L1 (e.g., a fusion protein containing the extracellular PD-1 binding portion of PD-L1 or PD-L2 fused to a constant region such as an Fc region of an immunoglobulin molecule). Examples of immunoadhesin molecules that specifically bind to PD-1 are described in US 2018/0169229. Specific fusion proteins useful as the PD-1 antagonist in the treatment methods, medicaments and uses of the present disclosure include AMP-224 (also known as B7-DCIg), which is a PD-L2-FC fusion protein and binds to human PD-1.

In some embodiments, the anti-human PD-1 monoclonal antibody or antigen-binding fragment comprises light chain CDRs of SEQ ID NOs:13, 14 and 15, and heavy chain CDRs of SEQ ID NOs:16, 17 and 18. In some embodiments, the anti-human PD-1 monoclonal antibody or antigen-binding fragment comprises: a light chain variable region comprising SEQ ID NO:19, and a heavy chain comprising SEQ ID NO:20. In some embodiments, the anti-human PD-1 monoclonal antibody comprises a light chain comprising SEQ ID NO:21, and a heavy chain comprising SEQ ID NO:22. In some embodiments, the anti-human PD-1 monoclonal antibody is pembrolizumab or a pembrolizumab variant.

In some embodiments, when the PD-1 antagonist is a mAb, the mAb may be a variant comprising heavy chain and light chain sequences that are identical to one of the anti-PD-1 or anti-PD-1L mAbs named in the preceding paragraphs (reference mAbs). The variant is identical to a reference mAb, except for having three, two or one conservative amino acid substitutions at positions that are located outside of the light chain complementarity determining regions (CDRs) and six, five, four, three, two or one conservative amino acid substitutions that are located outside of the heavy chain CDRs (e.g., the variant positions are located in the framework regions or the constant region). In other words, the reference mAb and the variant mAb comprise identical CDR sequences, but differ from each other due to having a conservative amino acid substitution at no more than three or six other positions in their full length light and heavy chain sequences, respectively. The variant mAb is substantially the same as the reference mAb with respect to the following properties: binding affinity to antigen and ability to block the binding of each of the antigen to a ligand.

As used herein the term “biosimilar” refers to a biological product that is similar to but without clinically meaningful differences in safety and effectiveness from a Federal Drug Administration (FDA)-approved reference product. For instance, there may be differences between a biosimilar product and a reference product in clinically inactive components (e.g., differences in excipients of the formulations, minor differences in glycosylation, etc.). Clinically meaningful characteristics can be assessed through pharmacokinetic and pharmacodynamic studies. In some embodiments, the biosimilar product is an interchangeable product as determined by the FDA.

### IV. Pharmaceutical Compositions

Some compositions of the present disclosure are pharmaceutical compositions comprising a pharmaceutically acceptable excipient and a TLR9 agonist. In some preferred embodiments, the TLR9 agonist is a CpG-C type ODN. Pharmaceutical compositions of the present disclosure may be in the form of a solution or a suspension. Alternatively, the pharmaceutical compositions may be a dehydrated solid (e.g., freeze dried or spray dried solid). The pharmaceutical compositions of the present disclosure are preferably sterile, and preferably essentially endotoxin-free. The term “pharmaceutical composition” is used interchangeably herein with the terms “medicinal product” and “medicament.”

In some embodiments, pharmaceutical compositions of the present disclosure comprise at least one excipient and a non-particulate TLR9 agonist (e.g., CpG-C type ODN). That is the TLR9 agonist is not present in a composition further comprising a tumor antigen and/or a multimerization agent. In some embodiments, the TLR9 agonist is not administered in combination with a tumor antigen and/or a biocompatible multimerization agent. As used herein, a “tumor antigen” is a polypeptide of about 9 to about 1000 amino acids, and a “multimerization agent” is a biocompatible compound with a diameter of about 10 to about 25,000 nanometers and/or a molecular weight of about 10,000 to about 1,000,000 Daltons. In some preferred embodiments, if a tumor antigen and/or a multimerization agent is present in a pharmaceutical composition comprising a TLR9 agonist, then both the TLR9 agonist and the tumor antigen are not both associated with the multimerization agent by one or more covalent linkages, or are not both associated with the multimerization agent by adsorption. As used herein, a multimerization agent may comprise either a polysaccharide having a diameter of from about 10 to about 1,000 nanometers and/or a molecular weight of about 10,000 to about 1,000,000 Daltons (e.g., a polysaccharide such as a branched copolymer of sucrose and epichlorohydrin, dextran, mannan, chitosan, agarose, or starch), or an aluminum hydroxide complex having a diameter of about 0.5 to about 25 micrometers (e.g., alum). Or said another way, in some embodiments, a pharmaceutical composition of the present disclosure is not an immunogenic composition described in WO 2017/181128.

A. Excipients

Pharmaceutically acceptable excipients of the present disclosure include for instance, solvents, bulking agents, buffering agents, tonicity adjusting agents, and preservatives (See, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration. That is the pharmaceutical compositions of the present disclosure are not intended for enteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

B. Kits

Additionally, the present disclosure provides kits that comprise a pharmaceutical composition (comprising an excipient and a CpG-C type oligonucleotide) and a set of instructions relating to the use of the composition for the methods describe herein. The pharmaceutical composition of the kits is packaged appropriately. For example, if the pharmaceutical composition is a freeze-dried power, a vial with a resilient stopper is normally used so that the powder may be easily resuspended by injecting fluid through the resilient stopper. In some embodiments, the kits further comprise a device for administration (e.g., syringe and needle, etc.) of the pharmaceutical composition. The instructions relating to the use of the pharmaceutical composition generally include information as to dosage, schedule and route of administration for the intended methods of use.

### V. Methods of Use

The pharmaceutical compositions of the present disclosure are suitable for treating cancer in a mammalian subject in need thereof. Mammalian subjects include but are not limited to humans, nonhuman primates, rodents, pets, and farm animals. In some embodiments, the pharmaceutical compositions may be administered to the subject in an amount effective to achieve a specific outcome.

A. Dosage and Mode of Administration

As with all pharmaceutical compositions, the effective amount and mode of administration may vary based on several factors evident to one skilled in the art. An important factor to be considered is whether the pharmaceutical composition is to be administered as a stand-alone treatment, or as part of a combination of therapeutic agents. Other factors to be considered include the outcome to be achieved, and the number of doses to be administered.

A suitable dosage range is one that provides the desired effect. Dosage may be determined by the amount of CpG-C type ODN to be administered to the subject. An exemplary dosage range of the oligonucleotide given in amount to be delivered by subject weight is from about 5 to 5000 mcg/kg. In some embodiments, the dosage is greater than about (lower limit) 5, 10, 50, 100, 150, 200, 250, 300, 350, 400, 450, 500, 750 or 1000 mcg/kg. In some embodiments, the dosage is less than about (upper limit) 5000, 4000, 3000, 2000, 1000, 750, 500, 450, 400, 350, 300, 250, 200, 150, or 100 mcg/kg. That is, the dosage is anywhere in the range of from about 5 to 5000 mcg/kg in which the lower limit is less than the upper limit. An exemplary dosage range of the oligonucleotide given in amount to be delivered to a subject is from about 100 mcg to about 100 mg. In some embodiments, the dosage is greater than about (lower limit) 100, 250, 500, 750, 1000, 1500, 2000, 2500, 3000, 3500, 4000, 4500, or 5000 mcg. In some embodiments, the dosage is less than about (upper limit) 100, 75, 50, 25, 20, 15, or 10 mg. That is, the dosage is anywhere in the range of from about 100 to 100,000 mcg in which the lower limit is less than the upper limit. In some embodiments, the dose is about 2 mg.

In some embodiments, the pharmaceutical compositions of the present disclosure are intended for parenteral administration (e.g., not oral or rectal administration). Suitable routes of administration include injection, topical, and inhalation. In particular, the pharmaceutical compositions of the present disclosure may be administered by a route such as intravenous, intramuscular, subcutaneous, epidermal (gene gun), transdermal, and inhalation. However, the preferred route of administration is by intratumoral delivery.

A suitable dosing regimen is one that provides the desired effect in a prophylactic or therapeutic context. The number of doses administered by a chosen route may be one or more than one. Frequency of dosing may range from every two, three, four, five or six days, weekly, bi-weekly, monthly, bi-monthly, or 3 to 12 months between doses. In some embodiments, 2 doses are administered with the second dose being administered one to two months after the first dose. In some embodiments, 3 doses are administered with the second dose being administered one to two months after the first dose, and the third dose being administered one to five months after the second dose. In other embodiments, 3, or 4 doses may be administered on a bi-weekly or monthly basis. In other embodiments, a shorter or longer period of time may elapse in between doses. In certain embodiments, the interval between successive dosages may vary in terms of number of weeks or number of months. In one embodiment, a series of 2, 3, 4, 5, or 6 weekly (or more frequent) doses may be administered followed by a second series of a number of weekly (or more frequent) doses at a later time point. One of skill in the art will be able to adjust the dosage regimen by measuring biological outcomes as exemplified in the examples, such as antigen-specific antibody responses or tumor regression.

B. Stimulation of an Immune Response

In brief, the present disclosure provides methods of stimulating an immune response in a mammalian subject, comprising administering to a mammalian subject a pharmaceutical composition in an amount sufficient to stimulate an immune response in the mammalian subject. “Stimulating” an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating interferon pathway-associated gene expression; stimulating chemoattractant-associated gene expression; and stimulating plasmacytoid dendritic cell (pDC) maturation.

Analysis (both qualitative and quantitative) of the immune response can be by any method known in the art, including, but not limited to, measuring activation of specific populations of lymphocytes such as cytotoxic T cells, production of cytokines such as IFN-alpha, IFN-gamma, IL-6, IL-12 and/or release of histamine. Activation of specific populations of lymphocytes can be measured by proliferation assays, and with fluorescence-activated cell sorting (FACS). Production of cytokines can also be measured by enzyme-linked immunosorbent assay (ELISA).

Preferably, a Th1-type immune response is stimulated (i.e., elicited or enhanced). With reference to present disclosure, stimulating a Th1-type immune response can be determined in vitro or ex vivo by measuring cytokine production from cells treated with an active agent of the present disclosure (TLR9 agonist) as compared to control cells not treated with the active agent. Examples of “Th1-type cytokines” include, but are not limited to, IL-2, IL-12, IFN-gamma and IFN-alpha. In contrast, “Th2-type cytokines” include, but are not limited to, IL-4, IL-5, and IL-13. Cells useful for the determination of immunostimulatory activity include cells of the immune system, such as antigen presenting cells and lymphocytes, preferably macrophages and T cells. Suitable immune cells include primary cells such as peripheral blood mononuclear cells isolated from a mammalian subject.

Stimulating a Th1-type immune response can also be determined in a mammalian subject treated with an active agent of the present disclosure (TLR9 agonist) by measuring levels of IL-2, IL-12, and interferon before and after administration or as compared to a control subject not treated with the active agent. Stimulating a Th1-type immune response can also be determined by measuring the ratio of Th1-type to Th2-type antibody titers. “Th1-type” antibodies include human IgG1 and IgG3, and murine IgG2a. In contrast, “Th2-type” antibodies include human IgG2, IgG4 and IgE and murine IgG1 and IgE.

C. Treating Cancer

The present disclosure provides methods of treating cancer in a mammalian subject in need thereof, comprising administering to the subject a CpG-C oligonucleotide and a PD-1 antagonist under conditions and together in an amount sufficient to treat cancer in the subject. “Treating” cancer means to bring about a beneficial clinical result such as causing remission or otherwise prolonging survival as compared to expected survival in the absence of treatment. In some embodiments, “treating” cancer comprises shrinking the size of a tumor or otherwise reducing viable cancer cell numbers. In other embodiments, “treating” cancer comprises delaying growth of a tumor. In some preferred embodiments, the present disclosure provides methods of treating cancer in a mammalian subject in need thereof, comprising administering to the subject a CpG-C oligonucleotide and a PD-1 antagonist together in an amount sufficient to treat cancer in the subject, in which the CpG-C oligonucleotide is administered by intratumoral delivery and the PD-1 antagonist is administered intravenously.

For instance, the present disclosure provides methods of inducing an antitumor response in a mammalian subject by administering to the mammalian subject a CpG-C oligonucleotide and a PD-1 antagonist together in an amount sufficient to induce an antitumor response in the subject. “Inducing” a tumor cell-specific CD8+ T-cell response means increasing the number of tumor-specific CD8+ T-cells above a threshold level such as a pre-treatment level. “Activating” a tumor cell-specific CD8+ T-cell response means increasing the number of antigen-specific CD8+ T-cells expressing surface markers of cellular activation, including granzyme B+, PD-llow, T-bet+, and the like.

In some preferred embodiments, “treating cancer” comprises assessing a patient's response to the treatment regimen according to the Response Evaluation Criteria in Solid Tumors (RECIST version 1.1) as described (see, e.g., Eisenhauer et al., Eur J Cancer, 45:228-247, 2009; and Nishino et al., Am J Roentgenol, 195: 281-289, 2010). Response criteria to determine objective anti-tumor responses per RECIST 1.1 include: complete response (CR); partial response (PR); progressive disease (PD); and stable disease (SD).

In some preferred embodiments, “treating cancer” comprises assessing a patient's response to the treatment regimen according to the Immunotherapy Response Evaluation Criteria in Solid Tumors (iRECIST) as described (see, e.g., Seymour et al., Lancet Oncol, 18(3):e143-e152, 2017). A significant difference between RECIST 1.1 and iRECIST is that iRECIST resets the bar when RECIST 1.1 progression is followed by tumor shrinkage. The Response criteria to determine objective anti-tumor responses per iRECIST include: immune complete response (iCR); immune complete progression (iCPD); immune partial response (iPR); immune stable disease (iSD); and immune unconfirmed progression (iUPD).

### VI. Exemplary Specific Embodiments

A. Embodiments Comprising a CpG-C type oligonucleotide in combination with a PD-1 antagonist and a taxane chemotherapeutic agent.

1. A method of treating breast cancer in a mammalian subject in need thereof, the method comprising administering to the subject a CpG-C type oligonucleotide in combination with a PD-1 antagonist and a taxane chemotherapeutic agent in a neoadjuvant regimen, wherein

the CpG-C type oligonucleotide comprises the sequence:

5′-TCNDyAACGTTCGAACGTTCGAANz-3′ (SEQ ID NO:2); or 5′-TCGDyAACGTTCGAACGTTCGAANz-3′ (SEQ ID NO:3), each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

2. The method according to embodiment 1, wherein the CpG-C type oligonucleotide comprises a sequence selected from the group consisting of:

(SEQ ID NO: 4)
5′-TCGAACGTTCGAACGTTCGAACGTTCGAAT-3′;
(SEQ ID NO: 5)
5′-TCGAACGTTCGAACGTTCGAACGTT-3′;
(SEQ ID NO: 6)
5′-TCGAACGTTCGAACGTTCGAATTTT-3′;
(SEQ ID NO: 7)
5′-TCGTAACGTTCGAACGTTCGAACGTTA-3′;
(SEQ ID NO: 8)
5′-TCGTAACGTTCGAACGTTCGAACGTT-3′;
(SEQ ID NO: 9)
5′-TCGTAACGTTCGAACGTTCGAACGT-3′;
(SEQ ID NO: 10)
5′-TCGTAACGTTCGAACGTTCGAACG-3′;
(SEQ ID NO: 11)
5′-TCGTAACGTTCGAACGTTCGAAC-3′;
and
(SEQ ID NO: 12)
5′-TCGTAACGTTCGAACGTTCGAA-3′.

3. The method according to embodiment 2, wherein the CpG-C type oligonucleotide comprises: 5′-TCGAACGTTCGAACGTTCGAACGTTCGAAT-3′ (SEQ ID NO:4).

4. The method according to embodiment 2, wherein all of the internucleotide linkages of the CpG-C type oligonucleotide are phosphorothioate ester linkages.

5. The method according to any one of embodiments 1-4, wherein the PD-1 antagonist is an anti-human PD-1 monoclonal antibody or an antigen-binding fragment thereof.

6. The method according to embodiment 5, wherein the anti-human PD-1 monoclonal antibody is selected from the group consisting of pembrolizumab, nivolumab, and cemiplimab.

7. The method according to embodiment 5, wherein the anti-human PD-1 monoclonal antibody or antigen-binding fragment comprises light chain CDRs of SEQ ID NOs:13, 14 and 15, and heavy chain CDRs of SEQ ID NOs:16, 17 and 18.

8. The method according to embodiment 5, wherein:

(i) the anti-human PD-1 monoclonal antibody or antigen-binding fragment comprises a light chain variable region comprising SEQ ID NO:19, and a heavy chain comprising SEQ ID NO:20; or

(ii) the anti-human PD-1 monoclonal antibody comprises a light chain comprising SEQ ID NO:21, and a heavy chain comprising SEQ ID NO:22.

9. The method according to any one of embodiments 1-4, wherein the PD-1 antagonist is an anti-human PD-L1 monoclonal antibody or antigen-binding fragment thereof.

10. The method according to embodiment 9, wherein the anti-human PD-L1 monoclonal antibody is selected from the group consisting of atezolizumab, durvalumab, and avelumab.

11. The method according to any one of embodiments 1-10, wherein the taxane chemotherapeutic agent is selected from the group consisting of paclitaxel, docetaxel, cabazitaxel, and albumin-bound paclitaxel.

12. The method according to embodiment 11, wherein the taxane chemotherapeutic agent is paclitaxel.

13. The method according to any one of embodiments 1-12, wherein the breast cancer is: (i) Stage II, Stage III, or Regional Stage IV, as staged according to the TNM system, or is T4, any N, and M0; or (ii) Stage II or Stage III.

14. The method according to embodiment 13, wherein the breast cancer is estrogen receptor and/or progesterone receptor negative.

15. The method according to embodiment 13, wherein the breast cancer is HER2-negative.

16. The method according to f embodiment 13, wherein the breast cancer is triple-negative (i.e., estrogen receptor-negative, progesterone receptor-negative, and HER2-negative).

17. The method according to any one of embodiments 1-16, wherein:

the CpG-C type oligonucleotide is administered by intratumoral injection at a dose of 2 mg;

the PD-1 antagonist is administered intravenously at a dose of 200 mg; and

the taxane chemotherapeutic agent is administered intravenously at a dose of 80 mg/m², optionally after steroid premedication.

18. The method according to embodiment 17, wherein:

the CpG-C type oligonucleotide is administered once a week (q1w) for three weeks and then once every three weeks (q3w) (i.e., weeks 1, 2, 3, 4, 7, and 10);

the PD-1 antagonist is administered once every three weeks (q3w) (i.e., weeks 1, 4, 7, and 10); and

the taxane chemotherapeutic agent is administered once a week (q1w) (i.e., weeks 1-12).

19. The method of embodiment 18, further comprising administering doxorubicin and cyclophosphamide after the neoadjuvant regimen with the combination of the CpG-C type oligonucleotide, the PD-1 antagonist and the taxane chemotherapeutic agent has been completed, wherein;

the doxorubicin is administered intravenously at a dose of 60 mg/m² once every two or three weeks (q2w or q3w) (i.e., weeks 15, 18, 21 and 24); and

the cyclophosphamide is administered intravenously at a dose of 600 mg/m² once every two or three weeks (q2w or q3w) (i.e., weeks 15, 18, 21 and 24).

20. The method according to any one of embodiments 1-19, wherein the mammalian subject is a human.

21. The method according to any one of embodiments 1-20, wherein the CpG-C type oligonucleotide is not associated with a multimerization agent by one or more covalent linkages, or by adsorption.

22. The method according to any one of embodiments 1-21, wherein the CpG-C type oligonucleotide is not administered together with an antigen, optionally wherein the antigen is a tumor antigen.

23. The method according to any one embodiments 1-22, wherein treating cancer results in a favorable outcome according to:

(i) response evaluation criteria in solid tumors version 1.1 (RECIST 1.1), and the favorable outcome comprises one or more of the following:

(a) a complete response of target lesion(s);

(b) a partial response of target lesion(s);

(c) a stable disease of target lesion(s);

(d) a complete response of non-target lesion(s); and

(e) a stable disease of non-target lesion(s); or

(ii) immunotherapy response evaluation criteria in solid tumors version (iRECIST), and the favorable outcome comprises one of the following:

(a) an immune complete response (iCR);

(b) an immune partial response (iPR);

(c) an immune stable disease (iSD); or

(d) an immune unconfirmed progression (iUPD).

24. The method according to any one of embodiments 1-22, wherein treating cancer results in a pathologic complete response (pCR) defined as an absence of invasive tumor in breast or lymph nodes of the subject at the completion of the neoadjuvant regimen.

25. The method according to any one of embodiments 1-22, wherein treating cancer comprises reducing size of target lesion(s) as measured using magnetic resonance imaging (MRI).

26. The method according to any one of embodiments 1-22, wherein treating cancer comprises lengthening time before breast cancer recurrence in the subject after surgical resection following completion of the neoadjuvant regimen, as compared to what was expected in the absence of administration of the CpG-C type oligonucleotide and the PD-1 antagonist.

27. A method of treating HER2-negative breast cancer in a mammalian subject in need thereof, the method comprising administering to the subject a CpG-C type oligonucleotide in combination with a PD-1 antagonist and a taxane chemotherapeutic agent in a neoadjuvant regimen, wherein

(i) the CpG-C type oligonucleotide is administered by intratumoral injection at a dose of 2 mg once a week (q1w) for three weeks and then once every three weeks (q3w) (i.e., weeks 1, 2, 3, 4, 7, and 10), the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which

each N is an independently selected nucleoside, D is G, A or T, y=0 or 1, z=0 to 19, and two or more internucleotide linkages are phosphorothioate ester linkages;

(ii) the PD-1 antagonist administered intravenously at a dose of 200 mg once every three weeks (q3w) (i.e., weeks 1, 4, 7, and 10), and the PD-1 antagonist is an anti-human PD-1 monoclonal antibody or an antigen-binding fragment thereof, or an anti-human PD-L1 monoclonal antibody or antigen-binding fragment thereof; and

(iii) the taxane chemotherapeutic agent is administered intravenously at a dose of 80 mg/m² once a week (q1w) (i.e., weeks 1-12) optionally after steroid premedication.

28. A composition comprising a CpG-C type oligonucleotide for use in a method of treating breast cancer in a mammalian subject in need thereof, the method comprising administering to the subject the CpG-C type oligonucleotide in combination with a PD-1 antagonist and a taxane chemotherapeutic agent in a neoadjuvant regimen, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which

each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

29. Use of a CpG-C type oligonucleotide in the manufacture of a medicament for use in a method of treating a mammalian subject diagnosed with breast cancer with the CpG-C type oligonucleotide in combination with a PD-1 antagonist and a taxane chemotherapeutic agent in a neoadjuvant regimen, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

30. Use of a CpG-C type oligonucleotide, a PD-1 antagonist and a taxane chemotherapeutic agent in the manufacture of medicaments for use in a method of treating a mammalian subject diagnosed with breast cancer, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

31. The method, composition or use of any one of embodiments 27-30, wherein

the CpG-C type oligonucleotide comprises the sequence of (SEQ ID NO:4);

the PD-1 antagonist is:

(i) an anti-human PD-1 monoclonal antibody or antigen-binding fragment comprising light chain complementarity determining regions (CDRs) of SEQ ID NOs:13, 14 and 15, and heavy chain CDRs of SEQ ID NOs:16, 17 and 18;

(ii) an anti-human PD-1 monoclonal antibody or antigen-binding fragment comprising a light chain variable region comprising SEQ ID NO:19, and a heavy chain comprising SEQ ID NO:20; or

(iii) an anti-human PD-1 monoclonal antibody comprising a light chain comprising SEQ ID NO:21, and a heavy chain comprising SEQ ID NO:22; and the taxane chemotherapeutic agent is paclitaxel.

B. Embodiments Comprising a CpG-C type oligonucleotide in combination with a PD-1 antagonist, in the absence of a taxane chemotherapeutic agent.

1. A method of treating breast cancer in a mammalian subject in need thereof, the method comprising administering to the subject a CpG-C type oligonucleotide in combination with a PD-1 antagonist, wherein

the CpG-C type oligonucleotide comprises the sequence:

5′-TCNDyAACGTTCGAACGTTCGAANz-3′ (SEQ ID NO:2); or

5′-TCGDyAACGTTCGAACGTTCGAANz-3′ (SEQ ID NO:3), in which each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

2. The method according to embodiment 1, wherein the CpG-C type oligonucleotide comprises a sequence selected from the group consisting of:

(SEQ ID NO: 4)
5′-TCGAACGTTCGAACGTTCGAACGTTCGAAT-3′;
(SEQ ID NO: 5)
5′-TCGAACGTTCGAACGTTCGAACGTT-3′;
(SEQ ID NO: 6)
5′-TCGAACGTTCGAACGTTCGAATTTT-3′;
(SEQ ID NO: 7)
5′-TCGTAACGTTCGAACGTTCGAACGTTA-3′;
(SEQ ID NO: 8)
5′-TCGTAACGTTCGAACGTTCGAACGTT-3′;
(SEQ ID NO: 9)
5′-TCGTAACGTTCGAACGTTCGAACGT-3′;
(SEQ ID NO: 10)
5′-TCGTAACGTTCGAACGTTCGAACG-3′;
(SEQ ID NO: 11)
5′-TCGTAACGTTCGAACGTTCGAAC-3′;
and
(SEQ ID NO: 12)
5′-TCGTAACGTTCGAACGTTCGAA-3′.

3. The method according to embodiment 2, wherein the CpG-C type oligonucleotide comprises: 5′-TCGAACGTTCGAACGTTCGAACGTTCGAAT-3′ (SEQ ID NO:4).

4. The method according to embodiment 2, wherein all of the internucleotide linkages of the CpG-C type oligonucleotide are phosphorothioate ester linkages.

5. The method according to any one of embodiments 1-4, wherein the PD-1 antagonist is an anti-human PD-1 monoclonal antibody or an antigen-binding fragment thereof.

6. The method according to embodiment 5, wherein the anti-human PD-1 monoclonal antibody is selected from the group consisting of pembrolizumab, nivolumab, and cemiplimab.

7. The method according to embodiment 5, wherein the anti-human PD-1 monoclonal antibody or antigen-binding fragment comprises light chain CDRs of SEQ ID NOs:13, 14 and 15, and heavy chain CDRs of SEQ ID NOs:16, 17 and 18.

8. The method according to embodiment 5, wherein:

(i) the anti-human PD-1 monoclonal antibody or antigen-binding fragment comprises a light chain variable region comprising SEQ ID NO:19, and a heavy chain comprising SEQ ID NO:20; or

(ii) the anti-human PD-1 monoclonal antibody comprises a light chain comprising SEQ ID NO:21, and a heavy chain comprising SEQ ID NO:22.

9. The method according to any one of embodiments 1-4, wherein the PD-1 antagonist is an anti-human PD-L1 monoclonal antibody or antigen-binding fragment thereof.

10. The method according to embodiment 9, wherein the anti-human PD-L1 monoclonal antibody is selected from the group consisting of atezolizumab, durvalumab, and avelumab.

11. The method according to any one of embodiments 1-10, wherein the breast cancer is: (i) Stage II, Stage III, or Regional Stage IV, as staged according to the TNM system, or is T4, any N, and M0; or (ii) Stage II or Stage III.

12. The method according to embodiment 11, wherein the breast cancer is estrogen receptor and/or progesterone receptor negative.

13. The method according to embodiment 11, wherein the breast cancer is HER2 negative.

14. The method according to embodiment 11, wherein the breast cancer is triple-negative (i.e., estrogen receptor-negative, progesterone receptor-negative, and HER2-negative).

15. The method according to any one of embodiments 1-14, wherein the treatment comprises a neoadjuvant regimen in which:

the CpG-C type oligonucleotide is administered by intratumoral injection at a dose of 2 mg; and

the PD-1 antagonist is administered intravenously at a dose of 200 mg.

16. The method according to embodiment 15, wherein:

the CpG-C type oligonucleotide is administered once a week (qlw) for three weeks and then once every three weeks (q3w) (i.e., weeks 1, 2, 3, 4, 7, and 10); and

the PD-1 antagonist is administered once every three weeks (q3w) (i.e., weeks 1, 4, 7, and 10).

17. The method according to any one of embodiments 1-16, wherein the mammalian subject is a human.

18. The method according to any one of embodiments 1-17, wherein the CpG-C type oligonucleotide is not associated with a multimerization agent by one or more covalent linkages, or by adsorption.

19. The method according to any one of embodiments 1-18, wherein the CpG-C type oligonucleotide is not administered together with an antigen, optionally wherein the antigen is a tumor antigen.

20. The method according to any one of embodiments 1-19, wherein treating cancer results in a favorable outcome according to:

(i) response evaluation criteria in solid tumors version 1.1 (RECIST 1.1), and the favorable outcome comprises one or more of the following:

(a) a complete response of target lesion(s);

(b) a partial response of target lesion(s);

(c) a stable disease of target lesion(s);

(d) a complete response of non-target lesion(s); and

(e) a stable disease of non-target lesion(s); or

(ii) immunotherapy response evaluation criteria in solid tumors version (iRECIST), and the favorable outcome comprises one of the following:

(a) an immune complete response (iCR);

(b) an immune partial response (iPR);

(c) an immune stable disease (iSD); or

(d) an immune unconfirmed progression (iUPD).

21. The method according to any one of embodiments 15-19, wherein treating cancer results in a pathologic complete response (pCR) defined as an absence of invasive tumor in breast or lymph nodes at the completion of the neoadjuvant regimen.

22. The method according to any one of embodiments 15-19, wherein treating cancer comprises reducing size of target lesion(s) as measured using magnetic resonance imaging (MRI).

23. The method according to any one of embodiments 15-19, wherein treating cancer comprises lengthening time before breast cancer recurrence in the subject after surgical resection following completion of the neoadjuvant regimen, as compared to what was expected in the absence of administration of the CpG-C type oligonucleotide and the PD-1 antagonist.

24. A method of treating HER2-negative breast cancer in a mammalian subject in need thereof, the method comprising administering to the subject a CpG-C type oligonucleotide in combination with a PD-1 antagonist in a neoadjuvant regimen, wherein

(i) the CpG-C type oligonucleotide is administered by intratumoral injection at a dose of 2 mg once a week (q1w) for three weeks and then once every three weeks (q3w) (i.e., weeks 1, 2, 3, 4, 7, and 10), the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which

each N is an independently selected nucleoside, D is G, A or T, y=0 or 1, z=0 to 19, and two or more internucleotide linkages are phosphorothioate ester linkages; and

(ii) the PD-1 antagonist administered intravenously at a dose of 200 mg once every three weeks (q3w) (i.e., weeks 1, 4, 7, and 10), and the PD-1 antagonist is an anti-human PD-1 monoclonal antibody or an antigen-binding fragment thereof, or an anti-human PD-L1 monoclonal antibody or antigen-binding fragment thereof.

25. A composition comprising a CpG-C type oligonucleotide for use in a method of treating breast cancer in a mammalian subject in need thereof, the method comprising administering to the subject the CpG-C type oligonucleotide in combination with a PD-1 antagonist in a neoadjuvant regimen, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which

each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

26. Use of a CpG-C type oligonucleotide in the manufacture of a medicament for use in a method of treating a mammalian subject diagnosed with breast cancer with the CpG-C type oligonucleotide in combination with a PD-1 antagonist and a taxane chemotherapeutic agent in a neoadjuvant regimen, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

27. Use of a CpG-C type oligonucleotide and a PD-1 antagonist in the manufacture of medicaments for use in a method of treating a mammalian subject diagnosed with breast cancer in a neoadjuvant regimen, wherein

the CpG-C type oligonucleotide comprises the sequence

5′-TCNDyAACGTTCGAACGTTCGAANz-3′ (SEQ ID NO:2); or

5′-TCGDyAACGTTCGAACGTTCGAANz-3′ (SEQ ID NO:3), in which each N is an independently selected nucleoside, D is G, A or T, y=0 or 1, z=0 to 19, and two or more internucleotide linkages are phosphorothioate ester linkages, and

the PD-1 antagonist is an anti-human PD-1 monoclonal antibody or an antigen-binding fragment thereof, or an anti-human PD-L1 monoclonal antibody or antigen-binding fragment thereof.

28. The method, composition or use of any one of embodiments 24-27, wherein

the CpG-C type oligonucleotide comprises the sequence of SEQ ID NO:4; and

the PD-1 antagonist is:

(i) an anti-human PD-1 monoclonal antibody or antigen-binding fragment comprising light chain complementarity determining regions (CDRs) of SEQ ID NOs:13, 14 and 15, and heavy chain CDRs of SEQ ID NOs:16, 17 and 18;

(ii) an anti-human PD-1 monoclonal antibody or antigen-binding fragment comprising a light chain variable region comprising SEQ ID NO:19, and a heavy chain comprising SEQ ID NO:20; or

(iii) an anti-human PD-1 monoclonal antibody comprising a light chain comprising SEQ ID NO:21, and a heavy chain comprising SEQ ID NO:22.

29. The method, composition or use of any one of embodiments 15-28, wherein the neoadjuvant regimen does not comprise administration of a taxane chemotherapeutic regimen.

### EXAMPLES

Abbreviations: AC (doxorubicin and cyclophosphamide); CDR (complementarity determining region); CpG (oligonucleotide including an unmethylated CG dinucleotide); CMPD (compound); CTRL (control); EC50 (half maximal effective concentration); ELISA (enzyme-linked immunosorbent assay); ER (estrogen receptor); FACS (fluorescence-activated cell sorting); HC (antibody heavy chain); HCVR (heavy chain variable region); HER2 (human epidermal growth factor receptor 2 (HER2); HR (hormone receptor); IFN-α (interferon-alpha); IFN-γ (interferon-gamma); iRECIST (immunotherapy); response evaluation criteria in solid tumors); IT (intratumoral); IV (intravenous); LC (antibody light chain); LCVR (light chain variable region); mcg or μg (microgram); MP (MammaPrint® breast cancer recurrence assay marketed by Agendia Inc.); MM (magnetic resonance imaging); ODN (oligodeoxynucleotide); PBMC (peripheral blood mononuclear cell); PBS (phosphate buffered saline); pCR (pathologic complete response); Pembro (pembrolizumab); PN (polynucleotide); PO (peroral); PR (progesterone receptor); RECIST (response evaluation criteria in solid tumors); TLR9 (Toll-like receptor 9); TNF-α (tumor necrosis factor-alpha); and WT (wild type).

Although, the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1. Oligonucleotide and Anti-Human PD-1 Monoclonal Antibody Sequences

This example describes exemplary CpG-C oligonucleotides for use in combination with a PD-1 antagonist and optionally paclitaxel for treating breast cancer. Table 1-1 shows the motifs and sequences of representative CpG-containing oligonucleotides, which are referred to herein interchangeably as CpGs or CpG-ODNs.

TABLE 1-1
CpG-Oligonucleotide Sequences^{∧}

| CMPD | Sequence (SEQ ID NO) |
|---|---|
| — | 5′-TCNq(X1X2CGX2′X1′CG)rNs-3′ (SEQ ID NO: 1) |
| | wherein each N is an independently selected nucleoside; |
| | X1 and X1′ are self-complementary and X2 and X2′ are |
| | self-complementary; q = 1 or 2; r = 1, 2, 3 or 4; and |
| | s = 1 to 29 |
| — | 5′-TCNDyAACGTTCGAACGTTCGAANz-3′ (SEQ ID NO: 2) |
| | wherein each N is an independently selected nucleoside; |
| | D is G, A or T; y = 0 or 1; and z = 0 to 19 |
| — | 5′-TCGDyAACGTTCGAACGTTCGAANz-3′ (SEQ ID NO: 3) |
| | wherein each N is an independently selected nucleoside; |
| | D is G, A or T; y = 0 or 1; and z = 0 to 19 |
| C69-01 | 5′-TCG AACGTTCGAACGTTCGAA CGTTCGAAT-3′ (SEQ ID NO: 4) |
| C69-02 | 5′-TCG AACGTTCGAACGTTCGAA CGTT-3′ (SEQ ID NO: 5) |
| C69-03 | 5′-TCG AACGTTCGAACGTTCGAA TTTT-3′ (SEQ ID NO: 6) |
| C69-04 | 5′-TCGTAACGTTCGAACGTTCGAA CGTTA-3′ (SEQ ID NO: 7) |
| C69-05 | 5′-TCGTAACGTTCGAACGTTCGAA CGTT-3′ (SEQ ID NO: 8) |
| C69-06 | 5′-TCGTAACGTTCGAACGTTCGAA CGT-3′ (SEQ ID NO: 9) |
| C69-07 | 5′-TCGTAACGTTCGAACGTTCGAA CG-3′ (SEQ ID NO: 10) |
| C69-08 | 5′-TCGTAACGTTCGAACGTTCGAA C-3′ (SEQ ID NO: 11) |
| C69-09 | 5′-TCGTAACGTTCGAACGTTCGAA -3′ (SEQ ID NO: 12) |
| C60-10 | 5′-TTCG AACGTTCGAACGTTCGAA T-3′ (SEQ ID NO: 23) |
| C69-11 | 5′-TTTTCG AACGTTCGAACGTTCGAA ATT-3′ (SEQ ID NO: 24) |

^{∧}Unless otherwise noted, the oligonucleotides are 2′-deoxyribopolynucleotides, and all internucleotide linkages are phosphorothioate ester linkages (i.e., phosphorothioate backbone).

This example also describes an exemplary anti-human PD-1 monoclonal antibody for use in combination with a CpG-C oligonucleotide and optionally paclitaxel for treating breast cancer. Table 1-2 shows complementarity determining region (CDR), variable region (VR), light chain (LC) and heavy chain (HC) sequences of pembrolizumab.

TABLE 1-2
Anti-Human PD-1 Monoclonal Antibody Sequences

| Region | Sequence (SEQ ID NO) |
|---|---|
| CDRL1 | RASKGVSTSGYSYLH (SEQ ID NO: 13) |
| CDRL2 | LASYLES (SEQ ID NO: 14) |
| CDRL3 | QHSRDLPLT (SEQ ID NO: 15) |
| CDRH1 | NYYMY (SEQ ID NO: 16) |
| CDRH2 | GINPSNGGTNFNEKFK (SEQ ID NO: 17) |
| CDRH3 | RDYRFDMGFDY (SEQ ID NO: 18) |
| LCVR | EIVLTQSPATLSLSPGERATLSCRASKGVSTSGYSYLHWYQQK |
| | PGQAPRLLIYLASYLESGVPARFSGSGSGTDFTLTISSLEPED |
| | FAVYYCQHSRDLPLTFGGGTKVEIK (SEQ ID NO: 19) |
| HCVR | QVQLVQSGVEVKKPGASVKVSCKASGYTFTNYYMYWVRQAPGQ |
| | GLEWMGGINPSNGGTNFNEKFKNRVTLTTDSSTTTAYMELKSL |
| | QFDDTAVYYCARRDYRFDMGFDYWGQGTTVTVSS (SEQ ID |
| | NO: 20) |
| LC | EIVLTQSPATLSLSPGERATLSCRASKGVSTSGYSYLHWYQQK |
| | PGQAPRLLIYLASYLESGVPARFSGSGSGTDFTLTISSLEPED |
| | FAVYYCQHSRDLPLTFGGGTKVEIKRTVAAPSVFIFPPSDEQL |
| | KSGTASVVCLLNNFYPREAKVQWKVDNALQSGNSQESVTEQDS |
| | KDSTYSLSSTLTLSKADYEKHKVYACEVTHQGLSSPVTKSFNR |
| | GEC (SEQ ID NO: 21) |
| HC | QVQLVQSGVEVKKPGASVKVSCKASGYTFTNYYMYWVRQAPGQ |
| | GLEWMGGINPSNGGTNFNEKFKNRVTLTTDSSTTTAYMELKSL |
| | QFDDTAVYYCARRDYRFDMGFDYWGQGTTVTVSSASTKGPSVF |
| | PLAPCSRSTSESTAALGCLVKDYFPEPVTVSWNSGALTSGVHT |
| | FPAVLQSSGLYSLSSVVTVPSSSLGTKTYTCNVDHKPSNTKVD |
| | KRVESKYGPPCPPCPAPEFLGGPSVFLFPPKPKDTLMISRTPE |
| | VTCVVVDVSQEDPEVQFNWYVDGVEVHNAKTKPREEQFNSTYR |
| | VVSVLTVLHQDWLNGKEYKCKVSNKGLPSSIEKTISKAKGQPR |
| | EPQVYTLPPSQEEMTKNQVSLTCLVKGFYPSDIAVEWESNGQP |
| | ENNYKTTPPVLDSDGSFFLYSRLTVDKSRWQEGNVFSCSVMHE |
| | ALHNHYTQKSLSLSLGK (SEQ ID NO: 22) |

### Example 2. Isolation and Stimulation of Human Leukocytes by Oligonucleotides

Activity of oligonucleotides (PN) was assessed in vitro by measurement of cytokine secretion by human peripheral blood mononuclear cells (PBMCs).

PBMCs were isolated from blood of healthy human donors using Ficoll-Paque. For IFN-α induction, duplicate cultures of PBMCs were incubated for 24 hours with increasing concentrations of oligonucleotides. IFN-α levels in cell culture supernatants were measured by ELISA (PBL Biomedical Laboratories, Piscataway, N.J.). The limit of maximal/minimal detection was 13160/102 pg/ml.

All oligonucleotides tested induced IFN-α production from human PBMCs over a broad concentration range. Higher maximum IFN-α levels were induced by C69-01 and C69-02 as compared to C69-10 and C69-11, although all four oligonucleotides had comparable IFN-α EC₅₀ values as shown in Table 2-1. Although all four oligonucleotides comprised the motif of SEQ ID NO:3, C69-10 and C69-11 included one and three extra nucleotides respectively at their 5′ ends as compared to the preferred motif of SEQ ID NO:3, C69-01 and C69-02.

TABLE 2-1
IFN-α Secretion by Human PBMC

| | | EC₅₀ | IFN-α |
|---|---|---|---|
| | | IFN-α | Max |
| CMPD | SEQUENCE (SEQ ID NO) | (μM) | (pg/ml) |
| — | 5′-TCGDyAACGTTCGAACGTTCGAANz-3′ (NO: 3) | — | — |
| C69-01 | 5′-TCG AACGTTCGAACGTTCGAACGTTCGAAT-3′ | 0.061 | 6030 |
| | (NO: 4) | | |
| C69-02 | 5′-TCG AACGTTCGAACGTTCGAACGTT-3′ (NO: 5) | 0.049 | 6008 |
| C69-10 | 5′-TTCG AACGTTCGAACGTTCGAAT-3′ (NO: 23) | 0.058 | 3820 |
| C69-11 | 5′-TTTTCG AACGTTCGAACGTTCGAAATT-3′ (NO: 24) | 0.065 | 1239 |

### Example 3. Phase 2 Clinical Trial of Intratumoral C69-01 in Combination with Pembrolizumab in Patients with Locally Advanced Breast Cancer

This example provides a description of a study comparing the efficacy of a CpG-C oligonucleotide and a PD-1 antagonist in combination with standard chemotherapy to the efficacy of standard chemotherapy alone in human breast cancer patients. The CpG-C oligonucleotide is C69-01, which is provided an aqueous solution containing 2 mg/ml C69-01 (manufactured by Dynavax Technologies Corporation, Berkeley, Calif.). The PD-1 antagonist is an anti-human PD-1 monoclonal antibody known as pembrolizumab, which is provided as an aqueous solution containing 100 mg/4 ml pembrolizumab (manufactured by Merck Sharp & Dohme Corporation, Rahway, N.J.).

The patient population includes women with histologically confirmed invasive cancer of the breast. Eligible patients have clinically or radiologically measureable disease, defined as the longest lesion diameter greater than or equal to 25 mm (2.5 cm). Eligible tumors must meet one of the following criteria: Stage II or III, or T4, any N, M0, including clinical or pathologic inflammatory cancer or Regional Stage IV, where supraclavicular lymph nodes are the only site of metastasis.

Pre-treatment breast cancer tissue samples are assessed for hormone receptor and growth factor receptor expression, as well as for expression of genes associated with distant metastasis. In particular, estrogen receptor (ER), progesterone receptor (PR), and human epidermal growth factor receptor 2 (HER2) expression of biopsy tissue is assessed by any one of several standard methods such as immunohistochemistry, fluorescence in situ hybridization, and microarray. As used herein, hormone receptor (HR) expression refers to ER and/or PR expression. In addition, to assess risk of distant metastasis within 5-10 years, biopsy tissue is subject to a MammaPrint® breast cancer recurrence assay (MP) marketed by Agendia Inc. (Irvine, Calif.). Patients with any estrogen receptor (ER), progesterone receptor (PR), and human epidermal growth factor receptor 2 (HER2) status are eligible to participate.

The first arm is available for randomization of patients who have HER2-negative tumors. Initially, a maximum of 75 patients are assigned to C69-01+Pembro 4 across the four HER2-negative subtypes: 1) HR−MP−, HR−MP+, HR+MP−, HR+MP+. For convenience this arm is referred to as C69-01+Pembro 4. In C69-01+Pembro 4, four cycles of pembrolizumab are added to paclitaxel in the first 12 weeks of neoadjuvant therapy, concurrent with weekly intralesional administration of C69-01 for the first 4 weeks, followed by intralesional injection of C69-01 every 3 weeks (q3w) for a total duration of 12 weeks (six cycles). Tumor volume is assessed by magnetic resonance imaging (MRI) at several times during the study: baseline, post-randomization (pre cycle 1), pre-AC treatment (pre-cycle 13) and pre-surgical resection (post cycle 16). After the first 12 weeks of therapy, all patients undergo biopsy before receiving four cycles of doxorubicin and cyclophosphamide (AC) every 2-3 weeks during the subsequent 8-12 weeks of neoadjuvant therapy.

A primary objective is to show non-inferiority of C69-01+Pembro 4 in comparison with the pathologic complete response (pCR) rates of other therapies in patients with HER2-negative tumors. pCR is defined as the absence of invasive tumor in breast or lymph nodes at the completion of all neoadjuvant chemotherapy. A primary analysis involves determination of probabilities of non-inferiority of the pCR rate of C69-01+Pembro 4 as compared with previously attainable pCR rates. The Bayesian probabilities are calculated in the usual way and include covariates HR, HER2, and MP. A co-primary analysis of C69-01+Pembro 4 involves a standard superiority comparison (graduation) of an experimental arm in the study with the control arm paclitaxel followed by AC in each of the four HER2-negative subtypes.

The dose schedule and regimen for neoadjuvant therapy is shown in Table 3-1 and Table 3-2. Intralesional (intratumoral) injection of C69-01 is to precede infusion of pembrolizumab. A window of at least 2-3 hours following the intralesional injection is to occur before premedications (including steroids) and infusion of paclitaxel.

TABLE 3-1
Neoadjuvant Therapy Schedule - Combination of Paclitaxel, C69-01 and
Pembrolizumab, Followed by Doxorubicin and Cyclophosphamide

| Agent | Dose | Route | Dose Regimen Week |
|---|---|---|---|
| C69-01 | 2 mg | Intralesional | 1, 2, 3, 4, 7 & 10 |
| | q1w for 4 cycles, then q3w for 2 | | |
| | cycles | | |
| Pembrolizumab | 200 mg | IV | 1, 4, 7 & 10 |
| | q3w for 4 cycles | | |
| Paclitaxel | 80 mg/m² | IV | 1-12 |
| | q1w for 12 cycles | | |
| Followed by | | | |
| Doxorubicin | 60 mg/m² | IV | 15, 18, 21 & 24 |
| | q2w or q3w for 4 cycles | | |
| Cyclophosphamide | 600 mg/m² | IV | 15, 18, 21 & 24 |
| | q2w or q3w for 4 cycles | | |

A fixed dose of 2 mg C69-01 is administered. A single lesion is injected, at a volume of 1 ml for tumors with a diameter less than 5.0 cm, or at a volume of 2 ml for tumors with a diameter greater than or equal to 5 cm. A 30-minute observation period, for post-injection local and systemic reactions, is required after the first injection of C69-01.

A fixed dose of 200 mg pembrolizumab is administered after the 30 minute observation period following C69-01 injection. Intravenous infusion of pembrolizumab is to take place during a 30 minute time period (−5 min/+10 min). −00

Steroid premedication is indicated before administration of paclitaxel to prevent severe hypersensitivity reactions. About 30 minutes prior to a first dose of paclitaxel and following pembrolizumab infusion, 10 mg dexamethasone is administered (IV or PO) with diphenhydramine HCl 25 to 50 mg (IV or PO). About 30 minutes prior to a second dose of paclitaxel, 6 mg dexamethasone is administered (IV or PO) with diphenhydramine HCl 25 to 50 mg (IV or PO). For patients who do not experience an infusion related hypersensitivity reaction with either the first or second doses of paclitaxel, subsequent steroid premedication is discontinued. Paclitaxel is administered by intravenous infusion over a suitable time period.

After completion of the C69-01, pembrolizumab and paclitaxel course, patients receive four cycles of doxorubicin plus cyclophosphamide every 2 weeks (q2w) or every 3 weeks (q3w) prior to surgical resection.

TABLE 3-2
Neoadjuvant Therapy Regimen - Combination of Paclitaxel, C69-01 and
Pembrolizumab, Followed by Doxorubicin and Cyclophosphamide (AC)

| | | | | | | | | | | | | | | | | |
|---|---|---|---|---|---|---|---|---|---|---|---|---|---|---|---|---|
| Cycle | 1 | 2 | 3 | 4 | 5 | 6 | 7 | 8 | 9 | 10 | 11 | 12 | 13 | 14 | 15 | 16 |
| Week | 1 | 2 | 3 | 4 | 5 | 6 | 7 | 8 | 9 | 10 | 11 | 12 | 15 | 18 | 21 | 24 |
| C69-01 | X | X | X | X | | | X | | | X | | | | | | |
| Pembrolizumab | X | | | X | | | X | | | X | | | | | | |
| Paclitaxel | X | X | X | X | X | X | X | X | X | X | X | X | | | | |
| AC | | | | | | | | | | | | | X | X | X | X |

Toxicity is graded for severity using the NCI Common Terminology Criteria for Adverse Events (CTCAE) version 4.0. Unless otherwise specified, C69-01 is held for any Grade 3 or higher drug-related adverse event (except for fatigue and clinically insignificant laboratory abnormalities). C69-01 treatment is resumed once the adverse event has resolved to Grade 0 or 1. C69-01 is also held for local injection-site reactions above Grade 1. C69-01 is also held for an absolute neutrophil count of <1000 cells/mm³.

1. A method of treating breast cancer in a mammalian subject in need thereof, the method comprising administering to the subject a CpG-C type oligonucleotide in combination with a PD-1 antagonist and a taxane chemotherapeutic agent in a neoadjuvant regimen, wherein
the CpG-C type oligonucleotide comprises the sequence 5′-TCNDyAACGTTCGAACGTTCGAANz-3′ (SEQ ID NO:2), each N is an independently selected nucleoside, D is G, A or T, y=0 or 1, z=0 to 19, and two or more internucleotide linkages are phosphorothioate ester linkages, and
the PD-1 antagonist is an anti-human PD-1 monoclonal antibody or an antigen-binding fragment thereof, or an anti-human PD-L1 monoclonal antibody or antigen-binding fragment thereof.

2. The method according to claim 1, wherein the CpG-C type oligonucleotide comprises a sequence selected from the group consisting of:
(SEQ ID NO: 4)
5′-TCGAACGTTCGAACGTTCGAACGTTCGAAT-3′;
(SEQ ID NO: 5)
5′-TCGAACGTTCGAACGTTCGAACGTT-3′;
(SEQ ID NO: 6)
5′-TCGAACGTTCGAACGTTCGAATTTT-3′;
(SEQ ID NO: 7)
5′-TCGTAACGTTCGAACGTTCGAACGTTA-3′;
(SEQ ID NO: 8)
5′-TCGTAACGTTCGAACGTTCGAACGTT-3′;
(SEQ ID NO: 9)
5′-TCGTAACGTTCGAACGTTCGAACGT-3′;
(SEQ ID NO: 10)
5′-TCGTAACGTTCGAACGTTCGAACG-3′;
(SEQ ID NO: 11)
5′-TCGTAACGTTCGAACGTTCGAAC-3′;
and
(SEQ ID NO: 12)
5′-TCGTAACGTTCGAACGTTCGAA-3′.

3. The method according to claim 2, wherein the CpG-C type oligonucleotide comprises:
(SEQ ID NO: 4)
5′-TCGAACGTTCGAACGTTCGAACGTTCGAAT-3′.

4. The method according to claim 2, wherein all of the internucleotide linkages of the CpG-C type oligonucleotide are phosphorothioate ester linkages.

5. The method according to claim 1, wherein the PD-1 antagonist is an anti-human PD-1 monoclonal antibody or an antigen-binding fragment thereof.

6. The method according to claim 5, wherein the anti-human PD-1 monoclonal antibody is selected from the group consisting of pembrolizumab, nivolumab, and cemiplimab.

7. The method according to claim 5, wherein the anti-human PD-1 monoclonal antibody or antigen-binding fragment comprises light chain complementarity determining regions (CDRs) of SEQ ID NOs:13, 14 and 15, and heavy chain CDRs of SEQ ID NOs:16, 17 and 18.

8. The method according to claim 5, wherein:
(i) the anti-human PD-1 monoclonal antibody or antigen-binding fragment comprises a light chain variable region comprising SEQ ID NO:19, and a heavy chain comprising SEQ ID NO:20; or
(ii) the anti-human PD-1 monoclonal antibody comprises a light chain comprising SEQ ID NO:21, and a heavy chain comprising SEQ ID NO:22.

9. The method according to claim 1, wherein the PD-1 antagonist is an anti-human PD-L1 monoclonal antibody or antigen-binding fragment thereof.

10. The method according to claim 9, wherein the anti-human PD-L1 monoclonal antibody is selected from the group consisting of atezolizumab, durvalumab, and avelumab.

11. The method according to claim 1, wherein the taxane chemotherapeutic agent is selected from the group consisting of paclitaxel, docetaxel, cabazitaxel, and albumin-bound paclitaxel.

12. The method according to claim 11, wherein the taxane chemotherapeutic agent is paclitaxel.

13. The method according to claim 1, wherein the breast cancer is:
(i) Stage II, Stage III, or Regional Stage IV, as staged according to the TNM system, or is T4, any N, and M0; or (ii) Stage II or Stage III.

14. The method according to claim 13, wherein the breast cancer is estrogen receptor and/or progesterone receptor negative.

15. The method according to claim 13, wherein the breast cancer is HER2-negative.

16. The method according to claim 13, wherein the breast cancer is triple-negative.

17. The method according to claim 1, wherein:
the CpG-C type oligonucleotide is administered by intratumoral injection at a dose of 2 mg;
the PD-1 antagonist is administered intravenously at a dose of 200 mg; and
the taxane chemotherapeutic agent is administered intravenously at a dose of 80 mg/m², optionally after steroid premedication.

18. The method according to claim 17, wherein:
the CpG-C type oligonucleotide is administered once a week (q1w) for three weeks and then once every three weeks (q3w);
the PD-1 antagonist is administered once every three weeks (q3w); and
the taxane chemotherapeutic agent is administered once a week (q1w).

19. The method of claim 18, further comprising administering doxorubicin and cyclophosphamide after the neoadjuvant regimen with the combination of the CpG-C type oligonucleotide, the PD-1 antagonist and the taxane chemotherapeutic agent has been completed, wherein;
the doxorubicin is administered intravenously at a dose of 60 mg/m² once every two or three weeks (q2w or q3w); and
the cyclophosphamide is administered intravenously at a dose of 600 mg/m² once every two or three weeks (q2w or q3w).

20. The method according to claim 1, wherein the mammalian subject is a human.

21. The method according to claim 1, wherein the CpG-C type oligonucleotide is not associated with a multimerization agent by one or more covalent linkages, or by adsorption.

22. The method according to claim 1, wherein the CpG-C type oligonucleotide is not administered together with an antigen, optionally wherein the antigen is a tumor antigen.

23. The method according to claim 1, wherein treating cancer results in a favorable outcome according to:
(i) response evaluation criteria in solid tumors version 1.1 (RECIST 1.1), and the favorable outcome comprises one or more of the following:
(a) a complete response of target lesion(s);
(b) a partial response of target lesion(s);
(c) a stable disease of target lesion(s);
(d) a complete response of non-target lesion(s); and
(e) a stable disease of non-target lesion(s); or
(ii) immunotherapy response evaluation criteria in solid tumors version (iRECIST), and the favorable outcome comprises one of the following:
(a) an immune complete response (iCR);
(b) an immune partial response (iPR);
(c) an immune stable disease (iSD); or
(d) an immune unconfirmed progression (iUPD).

24. The method according to claim 1, wherein treating cancer results in a pathologic complete response (pCR) defined as an absence of invasive tumor in breast or lymph nodes of the subject at the completion of the neoadjuvant regimen.

25. The method according to claim 1, wherein treating cancer comprises reducing size of target lesion(s) as measured using magnetic resonance imaging (MRI).

26. The method according to claim 1, wherein treating cancer comprises lengthening time before breast cancer recurrence in the subject after surgical resection following completion of the neoadjuvant regimen, as compared to what was expected in the absence of administration of the CpG-C type oligonucleotide and the PD-1 antagonist.

27. A method of treating HER2-negative breast cancer in a mammalian subject in need thereof, the method comprising administering to the subject a CpG-C type oligonucleotide in combination with a PD-1 antagonist and a taxane chemotherapeutic agent in a neoadjuvant regimen, wherein
(i) the CpG-C type oligonucleotide is administered by intratumoral injection at a dose of 2 mg once a week (q1w) for three weeks and then once every three weeks (q3w),
the CpG-C type oligonucleotide comprises the sequence 5′-TCNDyAACGTTCGAACGTTCGAANz-3′ (SEQ ID NO:2), in which each N is an independently selected nucleoside, D is G, A or T, y=0 or 1, z=0 to 19, and two or more internucleotide linkages are phosphorothioate ester linkages;
(ii) the PD-1 antagonist is administered intravenously at a dose of 200 mg once every three weeks (q3w), and the PD-1 antagonist is an anti-human PD-1 monoclonal antibody or an antigen-binding fragment thereof, or an anti-human PD-L1 monoclonal antibody or antigen-binding fragment thereof; and
(iii) the taxane chemotherapeutic agent is administered intravenously at a dose of 80 mg/m² once a week (q1w), optionally after steroid premedication.

28. A composition comprising a CpG-C type oligonucleotide for use in a method of treating breast cancer in a mammalian subject in need thereof, the method comprising administering to the subject the CpG-C type oligonucleotide in combination with a PD-1 antagonist and a taxane chemotherapeutic agent in a neoadjuvant regimen, wherein
the CpG-C type oligonucleotide comprises of the sequence 5′-TCNDyAACGTTCGAACGTTCGAANz-3′ (SEQ ID NO:2), in which each N is an independently selected nucleoside, D is G, A or T, y=0 or 1, z=0 to 19, and two or more internucleotide linkages are phosphorothioate ester linkages, and
the PD-1 antagonist is an anti-human PD-1 monoclonal antibody or an antigen-binding fragment thereof, or an anti-human PD-L1 monoclonal antibody or antigen-binding fragment thereof.

29. Use of a CpG-C type oligonucleotide in the manufacture of a medicament for use in a method of treating a mammalian subject diagnosed with breast cancer with the CpG-C type oligonucleotide in combination with a PD-1 antagonist and a taxane chemotherapeutic agent in a neoadjuvant regimen, wherein
the CpG-C type oligonucleotide comprises the sequence 5′-TCNDyAACGTTCGAACGTTCGAANz-3′ (SEQ ID NO:2), in which each N is an independently selected nucleoside, D is G, A or T, y=0 or 1, z=0 to 19, and two or more internucleotide linkages are phosphorothioate ester linkages, and
the PD-1 antagonist is an anti-human PD-1 monoclonal antibody or an antigen-binding fragment thereof, or an anti-human PD-L1 monoclonal antibody or antigen-binding fragment thereof.

30. Use of a CpG-C type oligonucleotide, a PD-1 antagonist and a taxane chemotherapeutic agent in the manufacture of medicaments for use in a method of treating a mammalian subject diagnosed with breast cancer, wherein
the CpG-C type oligonucleotide comprises the sequence: 5′-TCNDyAACGTTCGAACGTTCGAANz-3′ (SEQ ID NO:2), in which each N is an independently selected nucleoside, D is G, A or T, y=0 or 1, z=0 to 19, and two or more internucleotide linkages are phosphorothioate ester linkages, and
the PD-1 antagonist is an anti-human PD-1 monoclonal antibody or an antigen-binding fragment thereof, or an anti-human PD-L1 monoclonal antibody or antigen-binding fragment thereof.

31. The method of claim 27, the composition of claim 28, the use of claim 29, or the use of claim 30, wherein
the CpG-C type oligonucleotide comprises the sequence of SEQ ID NO:4;
the PD-1 antagonist is:
(i) an anti-human PD-1 monoclonal antibody or antigen-binding fragment comprising light chain complementarity determining regions (CDRs) of SEQ ID NOs:13, 14 and 15, and heavy chain CDRs of SEQ ID NOs:16, 17 and 18;
(ii) an anti-human PD-1 monoclonal antibody or antigen-binding fragment comprising a light chain variable region comprising SEQ ID NO:19, and a heavy chain comprising SEQ ID NO:20; or
(iii) an anti-human PD-1 monoclonal antibody comprising a light chain comprising SEQ ID NO:21, and a heavy chain comprising SEQ ID NO:22; and
the taxane chemotherapeutic agent is paclitaxel.


SEQUENCE LISTING (24 sequences)

SEQ ID NO: 1 - DNA, 13 bp, Artificial Sequence
  Synthetic Construct
  misc_feature (3)..(3): n = Any nucleoside
  misc_feature (4)..(4): n = Any nucleoside, but can be absent
  misc_feature (5)..(6): n = Any nucleoside, and position 5 and 6 are self-complementary
  misc_feature (9)..(10): n = Any nucleoside, and position 9 and 10 are self-complementary
  misc_feature (5)..(12): Can be present in repeats of 1, 2, 3 or 4
  misc_feature (13)..(13): n = An independently selected nucleoside, and can be present in repeats of 1, 2, 3, 4, 5, 6, 7, 8, 9, 10, 11, 12, 13, 14, 15, 16, 17, 18, 19, 20, 21, 22, 23, 24, 25, 26, 27, 28, or 29
tcnnnncgnncgn

SEQ ID NO: 2 - DNA, 23 bp, Artificial Sequence
  Synthetic Construct
  misc_feature 3: n = An independently selected nucleoside
  misc_feature 4: n = G, A or T, and can be absent or present
  misc_feature 23: n = An independently selected nucleoside, and can be present in repeats of 1, 2, 3, 4, 5, 6, 7, 8, 9, 10, 11, 12, 13, 14, 15, 16, 17, 18, or 19
tcnnaacgttcgaacgttcgaan

SEQ ID NO: 3 - DNA, 23 bp, Artificial Sequence
  Synthetic Construct
  misc_feature 4: n = G, A or T, and can be absent or present
  misc_feature 23: n = An independently selected nucleoside, and can be present in repeats of 1, 2, 3, 4, 5, 6, 7, 8, 9, 10, 11, 12, 13, 14, 15, 16, 17, 18, or 19
tcgnaacgttcgaacgttcgaan

SEQ ID NO: 4 - DNA, 30 bp, Artificial Sequence
  Synthetic Construct
tcgaacgttcgaacgttcgaacgttcgaat

SEQ ID NO: 5 - DNA, 25 bp, Artificial Sequence
  Synthetic Construct
tcgaacgttcgaacgttcgaacgtt

SEQ ID NO: 6 - DNA, 25 bp, Artificial Sequence
  Synthetic Construct
tcgaacgttcgaacgttcgaatttt

SEQ ID NO: 7 - DNA, 27 bp, Artificial Sequence
  Synthetic Construct
tcgtaacgttcgaacgttcgaacgtta

SEQ ID NO: 8 - DNA, 26 bp, Artificial Sequence
  Synthetic Construct
tcgtaacgttcgaacgttcgaacgtt

SEQ ID NO: 9 - DNA, 25 bp, Artificial Sequence
  Synthetic Construct
tcgtaacgttcgaacgttcgaacgt

SEQ ID NO: 10 - DNA, 24 bp, Artificial Sequence
  Synthetic Construct
tcgtaacgttcgaacgttcgaacg

SEQ ID NO: 11 - DNA, 23 bp, Artificial Sequence
  Synthetic Construct
tcgtaacgttcgaacgttcgaac

SEQ ID NO: 12 - DNA, 22 bp, Artificial Sequence
  Synthetic Construct
tcgtaacgttcgaacgttcgaa

SEQ ID NO: 13 - protein, 15 aa, Artificial Sequence
  Synthetic Construct
RASKGVSTSGYSYLH

SEQ ID NO: 14 - protein, 7 aa, Artificial Sequence
  Synthetic Construct
LASYLES

SEQ ID NO: 15 - protein, 9 aa, Artificial Sequence
  Synthetic Construct
QHSRDLPLT

SEQ ID NO: 16 - protein, 5 aa, Artificial Sequence
  Synthetic Construct
NYYMY

SEQ ID NO: 17 - protein, 16 aa, Artificial Sequence
  Synthetic Construct
GINPSNGGTNFNEKFK

SEQ ID NO: 18 - protein, 11 aa, Artificial Sequence
  Synthetic Construct
RDYRFDMGFDY

SEQ ID NO: 19 - protein, 111 aa, Artificial Sequence [sequence omitted]

SEQ ID NO: 20 - protein, 120 aa, Artificial Sequence [sequence omitted]

SEQ ID NO: 21 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - DNA, 23 bp, Artificial Sequence
  Synthetic Construct
ttcgaacgttcgaacgttcgaat

SEQ ID NO: 24 - DNA, 27 bp, Artificial Sequence
  Synthetic Construct
ttttcgaacgttcgaacgttcgaaatt